(19) United States
(12) Patent Application Publication
(10) Pub. No.: US 2008/0227130 A1
(43) Pub. Date: Sep. 18, 2008

(54) Method for detecting exposure to ionizing radiation

(76) Inventor: Tulsee Satish Doshi, Portland, OR (US)

Correspondence Address: Richard A. Wilhelm, PO Box 171, Marylhurst, OR, 97036, US

(21) Appl. No.: 12/075,695

(22) Filed: Mar. 12, 2008

Related U.S. Application Data
(60) Provisional application No. 60/918,464, filed on Mar. 15, 2007.

(51) Int. Cl. C12Q 1/54

(52) U.S. Cl. 435/14

(57) ABSTRACT

A method for detecting exposure to ionizing radiation. An HbA1c test is performed on blood extracted from an animal at a first time, to establish a baseline glycosylated hemoglobin score, and a second HbA1c test is performed on blood extracted from the animal at a second time, to establish a test glycosylated hemoglobin score. The test and baseline scores are compared to determine any difference, and an inference is drawn as to whether the animal has or has not been exposed to ionizing radiation between the first and second times based, at least in part, on the amount of the difference.

### CROSS-REFERENCE TO RELATED APPLICATION

This application claims the benefit of U.S. Provisional Patent Application Ser. No. 60/918,464 entitled “USING Hb1AC TEST TO DETECT EXPOSURE TO IONIZING RADIATION,” filed Mar. 15, 2006, and incorporated herein by reference in its entirety.

### FIELD OF THE INVENTION

The present invention relates to a method for detecting exposure to ionizing radiation, such as X-rays.

### BACKGROUND

The need for detecting and quantifying exposure to ionizing radiation, such as X-rays, is well known. In laboratory and medical settings, the risk of exposure is anticipated and at-risk personnel typically utilize film badges to monitor cumulative radiation dose. However, even low doses of ionizing radiation can be harmful and exposure is not always anticipated; therefore, the person or animal exposed may not be equipped with a radiation monitoring substance or device. In such cases, it may be important to be able to detect exposure based on tests that can be performed on the body of the person or animal. The need for detection has been recognized in the prior art. For example:

U.S. Pat. No. 7,084,628 discloses performing radiation dosimetry based on measurements of the teeth utilizing electron paramagnetic resonance (EPR) techniques.

U.S. Pat. No. 5,042,962 discloses the use of persistent biological indicators of exposure to ionizing radiation, particularly nucleic acids corresponding to a gene or gene fragment, utilizing the technique of differential display to identify changes in gene expression at various timepoints after in vitro irradiation of a cell culture.

Similarly, for the purpose of identifying compounds that modulate a cell's response to ultraviolet radiation exposure, U.S. Pat. No. 7,105,292, discloses methods for detecting the exposure comprising measuring the levels of a plurality of RNA or protein molecules in the cell and noting an increased or decreased level of expression of one or more nucleic acids.

It has also been recently noted (Science Daily) that specific gene profiles distinguish individuals that were exposed to radiation from those that were not exposed.

U.S. Pat. No. 6,132,981 analyzes blood cells to determine increases in a protein attached to the red blood cells, which is correlated with radiation exposure. The reference indicates that there are a number of such proteins that can be identified in a number of different ways.

All of these techniques require sophisticated equipment and analytical techniques that are costly and have limited availability. There remains a need for a test for radiation exposure that is more readily available and easier to perform.

### SUMMARY

A method for detecting exposure to ionizing radiation, such as X-rays, is disclosed herein. According to the method, an HbA1c test is performed on blood extracted from an animal at a first time, to establish a baseline glycosylated hemoglobin score, and a second HbA1c test is performed on blood extracted from the animal at a second time, to establish a test glycosylated hemoglobin score. The test and baseline scores are compared to determine any difference, and an inference is drawn as to whether the animal has or has not been exposed to ionizing radiation between the first and second times based, at least in part, on the amount of the difference.

It is to be understood that this summary is provided as a means of generally determining what follows in the drawings and detailed description and is not intended to limit the scope of the invention. Objects, features and advantages of the invention will be readily understood upon consideration of the following detailed description taken in conjunction with the accompanying drawings.

### DESCRIPTION OF DRAWINGS

FIG. 1 is a plot of increase in HbA1c score as a function of radiation dose.

### DETAILED DESCRIPTION OF PREFERRED EMBODIMENTS

According to the present invention, a common and relatively inexpensive blood test used in the diagnosis and treatment of diabetes has been found to provide results correlating with exposure to ionizing radiation, particularly X-rays.

The test is known as HbA1c, glycohemoglobin, glycated or glycosylated hemoglobin test (hereinafter “HbA1c test”). The HbA1c test indicates average blood glucose level, a level that is typically, in humans, within the range of about 4-8 percent. More specifically, the HbA1c test measures the percentage of hemoglobin molecules, in the red blood cells, that have glucose attached, i.e., glycohemoglobin (glycosylated hemoglobin), as compared to hemoglobin molecules that do not (non-glycosylated hemoglobin). The use of the HbA1c test has been described, e.g., in U.S. Pat. No. 4,649,122. The HbA1c test is a standard laboratory test conducted on blood samples extracted from the body as is well known.

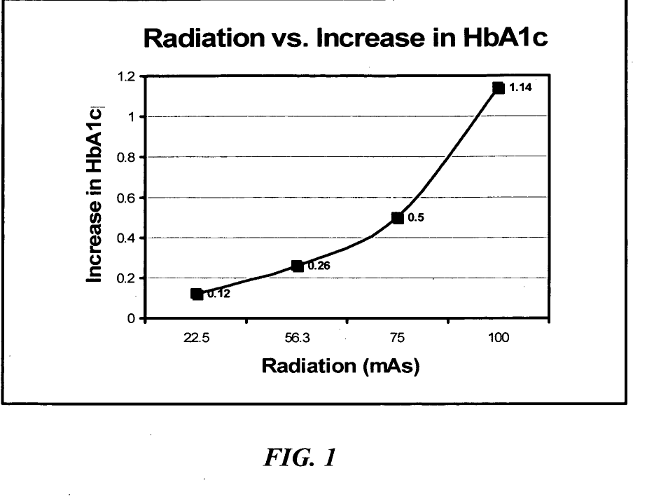

The present inventor has discovered that the HbA1c test can be used for detecting and quantifying exposure to at least some forms of ionizing radiation. More specifically, the relative proportions of glycosylated and non-glycosylated hemoglobin have been found to correlate positively with dosage of X-radiation, as shown in FIG. 1. FIG. 1 shows the change in HbA1c score as a function of radiation exposure produced by an X-ray machine (radiation exposure function). X-ray machines utilize vacuum tubes to produce X-rays, and the amount and energy of X-radiation produced by the X-ray machine is adjusted to provide varying dosages in units of tube filament current (milliamps) multiplied by time (seconds), i.e., milliamp-seconds or “mAs.” The intensity of the radiation is proportional to the filament current, and the time is the time during which radiation of the specified intensity is produced. The intensity is integrated over the time (or exposure), to define a total energy or dose emitted from the X-ray machine.

The actual radiation dose impacting a blood sample depends on the radiation pattern of the specific X-ray machine and the position of the blood sample relative to the X-ray machine, parameters which can be accounted for by standard calibration techniques. In the test results as represented in FIG. 1, no calibration was performed; however, all blood samples were placed at a fixed location relative to the X-ray machine, 40 cm distant therefrom. Forty blood samples taken from a random population of humans were exposed to the radiation. Table 1, below, shows the raw data.

TABLE 1

| | Before | After | Change | Before | After | Change |
|---|---|---|---|---|---|---|
| | 100 mAs, 80 kVp | | | 75 mAs, 80 kVp | | |
| | 7.7 | 9.1 | 1.4 | 6.9 | 7.4 | 0.5 |
| | 5.3 | 6.3 | 1.0 | 7.5 | 8.3 | 0.8 |
| | 6.1 | 6.9 | 0.8 | 6.8 | 7.0 | 0.2 |
| | 5.6 | 6.8 | 1.2 | 8.5 | 9.0 | 0.5 |
| | 7.0 | 8.1 | 1.1 | 7.8 | 8.1 | 0.3 |
| | 6.3 | 7.8 | 1.5 | 6.3 | 6.9 | 0.6 |
| | 7.3 | 8.7 | 1.4 | 6.5 | 6.9 | 0.4 |
| | 6.3 | 7.4 | 1.1 | 6.8 | 7.4 | 0.6 |
| | 5.5 | 6.3 | 0.8 | 6.9 | 7.3 | 0.4 |
| | 6.5 | 7.6 | 1.1 | 5.9 | 6.6 | 0.7 |
| Avg. Chg. | | | 1.14 | | | 0.5 |
| Std. Dev. | | | 0.24 | | | 0.18 |
| | 56.3 mAs, 80 kVp | | | 22.5 mAs, 80 kVp | | |
| | 6.1 | 6.3 | 0.2 | 4.2 | 4.4 | 0.2 |
| | 7.1 | 7.6 | 0.5 | 5.9 | 6.1 | 0.2 |
| | 6.2 | 6.5 | 0.3 | 7.4 | 7.5 | 0.1 |
| | 6.1 | 6.4 | 0.3 | 4.8 | 5.1 | 0.3 |
| | 6.0 | 6.4 | 0.4 | 4.9 | 5.0 | 0.1 |
| | 5.6 | 5.8 | 0.2 | 7.7 | 7.6 | −0.1 |
| | 5.9 | 6.2 | 0.3 | 6.5 | 6.7 | 0.2 |
| | 6.1 | 6.2 | 0.1 | 6.3 | 6.6 | 0.3 |
| | 5.1 | 5.3 | 0.2 | 6.1 | 6.0 | −0.1 |
| | 6.8 | 6.9 | 0.1 | 6.8 | 6.8 | 0.0 |
| Avg. Chg. | | | 0.26 | | | 0.12 |
| Std. Dev. | | | 0.13 | | | 0.15 |

The method is performed by applying the HbA1c test both before and after a known event, suspected event, or possible event of exposure to the ionizing radiation. Due to the ubiquitous availability and low cost of the HbA1c test, it is practical, and may be advantageous apart from its benefits as a radiation detecting mechanism, for all persons to have on file with their health care professional an HbA1c test result, which may be updated periodically, such as every year. This one-time or periodic testing establishes one or more “baseline” HbA1c test results, or one or more baseline HbA1c “scores.”

Then, after a suspected exposure to ionizing radiation has occurred, another HbA1c test is performed to obtain an HbA1c “test” score, for comparison with the baseline results. The difference between the baseline score and the test score is indicative of exposure to ionizing radiation.

FIG. 1 shows a function relating change in HbA1c score, on the vertical axis, to radiation dose as indicated on the horizontal axis, it being understood that the radiation dose as indicated is proportional to the actual radiation dose. For example, a radiation dose of 75 mAs produced an increase in HbA1c score of 0.5; so that blood from a person having a baseline HbA1c score of 6.0 (percent) would score 6.5 (percent) after being irradiated.

Glucose forms strong covalent bonds with hemoglobin, so once hemoglobin is glycated, it is expected to remain so; however, red blood cells die within a period of about 120 days, and at any given time of exposure, red blood cells of various ages will be present. Therefore, it is anticipated that the strength of the positive correlation between radiation dose and HbA1c score indicated in FIG. 1 would decay over time until reaching zero at about 120 days. The elapsed time between radiation exposure and the performance of the HbA1c test in FIG. 1 was less than 1 hour.

It is believed that HbA1c score shows a strong positive correlation with radiation dose because, again, glucose forms strong covalent bonds with hemoglobin. The strong covalent bonds may render glycosylated hemoglobin more robust and less sensitive to destruction by the radiation than non-glycosylated hemoglobin. In addition, removal of electrons from non-glycosylated hemoglobin due to ionizing radiation may make non-glycosylated hemoglobin molecules less stable. Either possible mechanism would tend to increase the proportion of glycosylated to non-glycosylated hemoglobin. However, it is also understood that hemoglobin chemistry is complex, and there may well be other explanations for the results; e.g., ionizing radiation may act as a catalyst for the glycation of hemoglobin by dislodging electrons at the ends of the hemoglobin needed for attachment of the glucose.

Regardless of their explanation, the results shown in FIG. 1 indicate a strong correlation that can be used in practice to indicate both the occurrence and the amount of X-radiation exposure utilizing an HbA1c test. Moreover, exposure of a blood samples to other forms of ionizing radiation of the same or higher energy, such as gamma rays, alpha particles, and beta particles, may be expected to produce a similar if not stronger effect on the HbA1c count.

It is recognized that a change in HbA1c score could result from either a change in diabetic condition or radiation. It is known that the HbA1c test averages the blood glucose level over a period of about 2-3 months, and the effect of radiation exposure on the blood is expected to have a similar lifetime as indicated above. Therefore, while the HbA1c test by itself is sufficient to indicate a lack of exposure to ionizing radiation, it may be desired to supplement the HbA1c test with other standard blood tests, or other tests on the animal, to confirm the cause of the change in score. For example, another blood test may be utilized to indicate whether the animals' blood sugar level has changed, providing an independent indication of a possible change in diabetic condition, and blood testing can also indicate a change in white cell count which is independently indicative of exposure to radiation. Alternatively, any known standard test for radiation exposure, such as those indicated in the Background herein, may be utilized to confirm an initial diagnosis in the event the HbA1c test indicates a change in score and it is not readily apparent from circumstances which of the two causes is likely to be in operation.

Moreover, a mere suspicion of radiation exposure, under circumstances in which such can be considered likely to have occurred, may be sufficient to justify a treatment for radiation sickness. For example, a person may have visited a nuclear laboratory that, inadvertently, failed to provide the person with a radiation badge Such an event would be cause for inferring some likelihood of exposure to ionizing radiation, justifying blood testing according to the present invention.

It should be noted that the functional dependence shown in FIG. 1 is for ionizing radiation having a known photon energy. Where the photon energy of the ionizing radiation is known and a radiation function is constructed utilizing data corresponding to the same photon energy, direct comparison can be made to infer dosage. For example, a measured change in HbA1c score of +0.5 indicates the dose that is associated with a 75 mAs setting of the X-ray machine. However, it is not essential that the photon energy match the photon energy of the ionizing radiation to which the animal is exposed for meaningful comparisons to be made. Given the sensitivity of the HbA1c score indicated in FIG. 1, for example, it may be estimated how this would change if photon energy were increased or decreased. Moreover, any sudden and significant change in score is indicative of at least a likelihood of radiation exposure, without reference to a comparison to a radiation function such as shown in FIG. 1.

The terms and expressions which have been employed in the foregoing specification are used therein as terms of description and not of limitation, and there is no intention in the use of such terms and expressions to exclude equivalents of the features shown and described or portions thereof, it being recognized that the scope of the invention is defined and limited only by the claims which follow

1. A method for detecting exposure to first ionizing radiation, comprising:
performing a first HbA1c test on blood extracted from an animal at a first time, to establish a baseline glycosylated hemoglobin score;
performing a second HbA1c test on blood extracted from said animal at a second time subsequent to said first time, to establish a test glycosylated hemoglobin score;
comparing said test score to said baseline score and to determine a difference therebetween; and
inferring whether the animal has or has not been exposed to the first ionizing radiation between said first and second times based at least in part on the amount of said difference.

2. The method of claim 1, further comprising quantifying the amount of first ionizing radiation received by said animal by comparing said difference between said test score and said baseline score to a radiation exposure function relating radiation dosage to HbA1c score.

3. The method of claim 2, wherein said radiation exposure function relates radiation dosage to changes in HbA1c score.

4. The method of claim 3, further comprising formulating said radiation exposure function using ionizing radiation known to have substantially the same photon energy as said first ionizing radiation.

5. The method of claim 4, further comprising, in response to an event, inferring a likelihood of exposure to the first ionizing radiation, wherein said second HbA1c test is performed within 120 days of said event.

6. The method of claim 3, further comprising, in response to an event, inferring a likelihood of exposure to the first ionizing radiation, wherein said second HbA1c test is performed within 120 days of said event.

7. The method of claim 2, further comprising, in response to an event, inferring a likelihood of exposure to the first ionizing radiation, wherein said second HbA1c test is performed within 120 days of said event.

8. The method of claim 1, further comprising, in response to an event, inferring a likelihood of exposure to the first ionizing radiation, wherein said second HbA1c test is performed within 120 days of said event.
